(19) United States
(12) Patent Application Publication
(10) Pub. No.: US 2016/0302670 A1
(43) Pub. Date: Oct. 20, 2016

(54) PHOTOACOUSTIC IMAGING APPARATUS AND METHOD OF CONTROLLING THE SAME

(71) Applicant: SAMSUNG ELECTRONICS CO., LTD., Suwon-si (KR)

(72) Inventors: Su Hyun PARK, Hwaseong-si (KR); Sung Chan PARK, Suwon-si (KR); Jung Ho KIM, Yongin-si (KR); Jong Keun SONG, Yongin-si (KR)

(73) Assignee: SAMSUNG ELECTRONICS CO., LTD., Suwon-si (KR)

(21) Appl. No.: 15/102,030

(22) PCT Filed: Dec. 4, 2014
(86) PCT No.: PCT/KR2014/011841

§ 371 Date: Jun. 6, 2016

(30) Foreign Application Priority Data
Dec. 4, 2013 (KR) 10-2013-0150157

(51) Int. Cl. A61B 5/00

(52) U.S. Cl. CPC A61B 5/0095; A61B 5/7278; A61B 5/742; A61B 2560/0437

(57) ABSTRACT

Disclosed herein are a photoacoustic imaging apparatus to project a continuous-wave laser beam onto an object to generate a photoacoustic image, and a method of controlling the same.

The photoacoustic imaging apparatus include a laser source to generate a continuous-wave (CW) laser beam, a deflection mirror to reflect the CW laser beam to the object while rotating, a transducer to collect acoustic waves generated in the object by the CW laser beam, and an image processor to generate a photoacoustic image based on the collected acoustic waves.

### TECHNICAL FIELD

Embodiments of the present invention relate to a photoacoustic imaging apparatus to generate a photoacoustic image by receiving acoustic waves generated in a material that absorbs laser light and a method of controlling the same.

### BACKGROUND ART

Medical imaging apparatuses are apparatuses to acquire an image of an object using transmission, absorption or reflection properties of ultrasonic waves, laser beams, X-rays, or the like with respect to the object and uses the image for diagnosis. Examples of the medical imaging apparatuses include ultrasonic imaging apparatuses, photoacoustic imaging apparatuses, X-ray imaging apparatuses, and the like.

Photoacoustic imaging is a method of noninvasively obtaining an internal image of an object using a photoacoustic effect and the photoacoustic effect refers to a phenomenon in which a material absorbs light or electromagnetic waves to generate an acoustic wave.

### DISCLOSURE OF INVENTION

### Technical Problem

Therefore, it is an aspect of the present invention to provide a photoacoustic imaging apparatus that projects a continuous wave laser beam onto an object to generate a photoacoustic image and a method of controlling the same.

### Solution to Problem

In accordance with one aspect of the present invention, a photoacoustic imaging apparatus includes a laser source to generate a continuous-wave laser beam, a deflection mirror to reflect the continuous-wave laser beam toward an object while rotating, a transducer to collect acoustic waves generated in the object by the continuous-wave laser beam, and an image processor to generate a photoacoustic image based on the collected acoustic waves.

In accordance with another aspect of the present invention, a method of controlling a photoacoustic imaging apparatus includes generating a continuous-wave laser beam, reflecting the continuous-wave laser beam toward the object by projecting the continuous-wave laser beam onto a rotating deflection mirror, collecting acoustic waves generated in the object by the continuous-wave laser beam, and generating a photoacoustic image based on the collected acoustic waves.

### Advantageous Effects of Invention

According to the photoacoustic imaging apparatus and the method of controlling the same according to another embodiment of the present invention, information, the photoacoustic image may include more information regarding the depth of the object by modulating frequency of the CW laser beam and projecting the frequency modulated continuous-wave laser beam onto the object.

### BRIEF DESCRIPTION OF DRAWINGS

These and/or other aspects of the invention will become apparent and more readily appreciated from the following description of the embodiments, taken in conjunction with the accompanying drawings of which:

FIG. 1 is a perspective view illustrating a photoacoustic imaging apparatus according to an embodiment of the present invention;

FIG. 2 is a control block diagram illustrating a photoacoustic imaging apparatus according to an embodiment of the present invention;

FIG. 3A illustrates a single laser diode according to an embodiment of the present invention;

FIG. 3B illustrates an array of a plurality of laser diodes according to an embodiment of the present invention;

FIGS. 4A and 4B are graphs illustrating energy change of laser beams with respect to time;

FIGS. 5A to 5C are diagrams for describing a method of controlling a continuous-wave laser beam to have an energy waveform similar to that of a pulsed laser beam using a deflection mirror;

FIG. 6 is a graph illustrating energy change of a continuous-wave laser beam projected using a deflection mirror with respect to time;

FIGS. 7A and 7B are diagrams illustrating photoacoustic probes according to embodiments of the present invention;

FIG. 8 is a control block diagram illustrating a photoacoustic imaging apparatus according to another embodiment of the present invention;

FIGS. 9A to 9C are diagrams illustrating various arrangements of a light delivery unit and a transducer on a photoacoustic probe;

FIG. 10A illustrates an external appearance of the light delivery element;

FIG. 10B illustrate an inner structure of the light delivery element;

FIG. 11 is a control block diagram illustrating a photoacoustic imaging apparatus according to another embodiment of the present invention;

FIGS. 12A and 12B are graphs for describing energy waveforms of frequency modulated continuous-wave laser beams;

FIGS. 13A to 13C are diagrams for describing a method of applying weights to photoacoustic images according to an embodiment of the present invention;

FIG. 14 is diagrams for describing a method of creating a synthetic photoacoustic image;

FIG. 15 is a flowchart illustrating a method of generating a photoacoustic image by projecting a CW laser beam according to an embodiment of the present invention; and

FIG. 16 is a flowchart illustrating a method of generating a photoacoustic image by projecting a CW laser beam according to another embodiment of the present invention.

### BEST MODE FOR CARRYING OUT THE INVENTION

Reference will now be made in detail to the embodiments of the present invention, examples of which are illustrated in the accompanying drawings, wherein like reference numerals refer to like elements throughout.

Hereinafter, a photoacoustic imaging apparatus and a method of controlling the same will be described with reference to the accompanying drawings.

Photoacoustic imaging, as a medical imaging technology for diagnosing an object, has been developed based on a combination of ultrasonic properties and photoacoustic properties of the object and has been utilized in a variety of diagnosis fields.

Photoacoustic imaging (PAI) is a technology suitable for imaging of biological tissues by combining a high spatial resolution of an ultrasonic image and a high contrast ratio of an optical image. When a laser beam having a nanoscale short wavelength is projected onto biological tissues, a short electromagnetic pulse of the laser beam is absorbed by the biological tissues and causes thermo-elastic expansion in the biological tissues, which act as an initial ultrasonic source, so as to generate a momentary acoustic wave. In general, the acoustic wave is an ultrasonic wave. Ultrasonic waves formed described above reach surfaces of the biological tissues with various delays and a photoacoustic image is obtained by using the same.

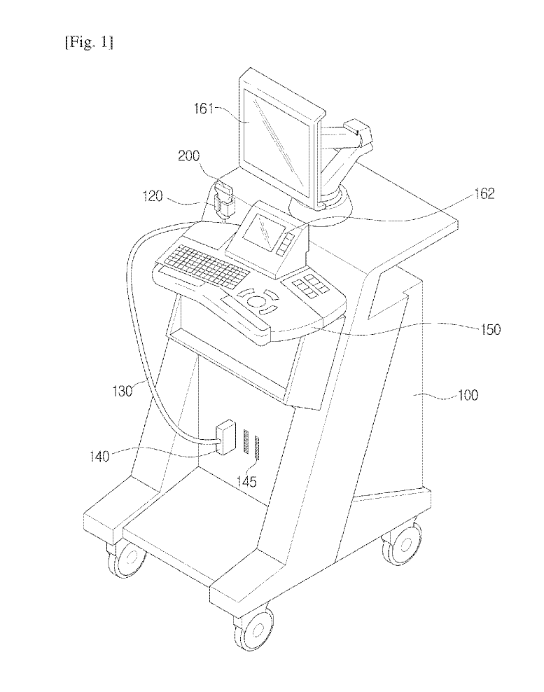

FIG. 1 is a perspective view illustrating a photoacoustic imaging apparatus according to an embodiment of the present invention. Referring to FIG. 1, the photoacoustic imaging apparatus may include a main body 100, a photoacoustic probe 200, an input unit 150, and a display 160.

The main body 100 may be provided with at least one female connector 145 at one side thereof. A male connector 140 connected to a cable 130 may be physically coupled to the female connector 145.

Meanwhile, a plurality of casters (not shown) may be provided at the bottom of the main body 100 to allow the photoacoustic imaging apparatus to move. The plurality of casters may fix the photoacoustic imaging apparatus at a predetermined place or allow the photoacoustic imaging apparatus to move in a predetermined direction.

The photoacoustic probe 200 which contacts the body surface of the object may receive acoustic waves. The photoacoustic probe 200 of FIG. 1 may include a laser source 210 and a deflection mirror 220 and may project a laser beam onto the object and receive corresponding acoustic waves. However, the laser source 210 and the deflection mirror 220 may also be separately formed from the photoacoustic probe 200.

The display 160 may include a main display 161 and a sub display 162.

The sub display 162 may be provided at the main body 100. FIG. 1 illustrates that the sub display 162 is provided on the input unit 150. The sub display 162 may display an application related to operation of a photoacoustic image apparatus. For example, the sub display 162 may display menus, guidelines, or the like for photoacoustic diagnosis. Examples of the sub display 162 may include cathode ray tubes (CRTs) and liquid crystal displays (LCDs).

The main display 161 may be provided at the main body 100. FIG. 1 illustrates that the main display 161 is provided at an upper portion than the sub display 162. The main display 161 may display a photoacoustic image acquired during the photoacoustic diagnosis. The main display 161 may also be a CRT or a LCD similarly to the sub display 162. Although FIG. 1 illustrates that the main display 161 is coupled to the main body 100, the main display 161 may also be separately formed from the main body 100.

FIG. 1 illustrates that the photoacoustic imaging apparatus includes both the main display 161 and the sub display 162. However, the sub display 162 may be dispensed with. In this case, the application or menus displayed on the sub display 162 may be displayed on the main display 161.

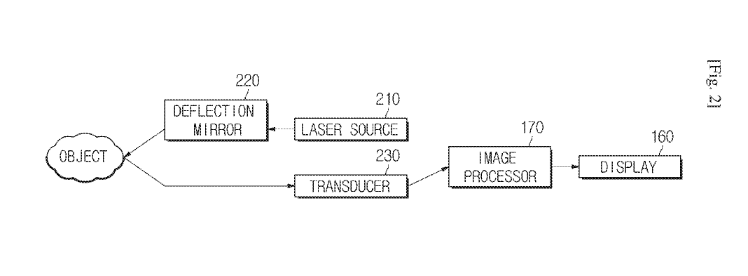

FIG. 2 is a control block diagram illustrating a photoacoustic imaging apparatus according to an embodiment of the present invention. The photoacoustic imaging apparatus may include a laser source 210 to generate a continuous-wave (CW) laser beam, a deflection mirror 220 to reflect the CW laser beam toward the object while rotating, a transducer 230 to collect acoustic waves generated in the object by the CW laser beam, and an image processor 170 to generate a photoacoustic image based on the collected acoustic waves. In FIG. 2, the laser source 210, the deflection mirror 220, and the transducer 230 are separately formed.

The laser source 210 may generate a CW laser beam. In order to generate the CW laser beam, the laser source 210 may include a single laser diode or a laser diode array in which a plurality of laser diodes are arranged in a predetermined direction.

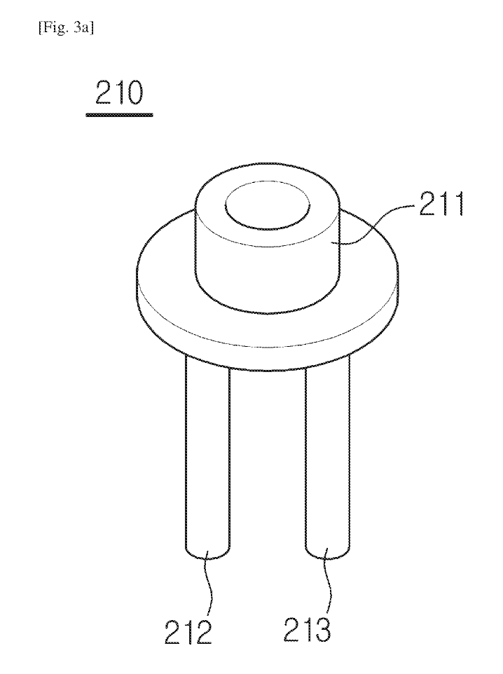

FIG. 3A illustrates a single laser diode according to an embodiment of the present invention. The laser diode is a diode that generates a laser beam when a forward current is supplied to a PN junction.

As illustrated in FIG. 3A, the laser diode may include a positive electrode 212, a negative electrode 213, and a light emitting unit 211. The light emitting unit 211 that generates laser beams may include a PN junction. When current is supplied to the positive electrode 212, the light emitting unit 211 may generate a laser beam while the current flows to the negative electrode 213. Here, the generated laser beam may be a CW laser beam.

FIG. 3B illustrates an array of a plurality of laser diodes according to an embodiment of the present invention. The laser diodes may be arranged in a predetermined direction to form a laser diode array 214. To this end, the plurality of laser diodes may be arranged as a bar shape.

When the laser diode array 214 is used instead of the single laser diode, a plurality of laser beams may be emitted by a single process, so that more information may be acquired from the object.

FIG. 3B exemplarily illustrates a one-dimensional array of the plurality of laser diodes, but two-dimensional arrays thereof may also be used.

The deflection mirror 220 may reflect the CW laser beam toward the object while rotating. The deflection mirror 220 may include a reflective portion 221 to reflect the CW laser beam, a rotation portion 222 to transfer rotational force to the reflective portion 221, and a supporting portion 223 to support the rotation portion 222.

The deflection mirror 220 may control the CW laser beam generated by the laser source 210 to have an energy waveform similar to that of a pulsed laser beam.

Hereinafter, a method of controlling the CW laser beam to have an energy waveform similar to that of a pulsed laser beam using the deflection mirror 220 and grounds therefor will be described.

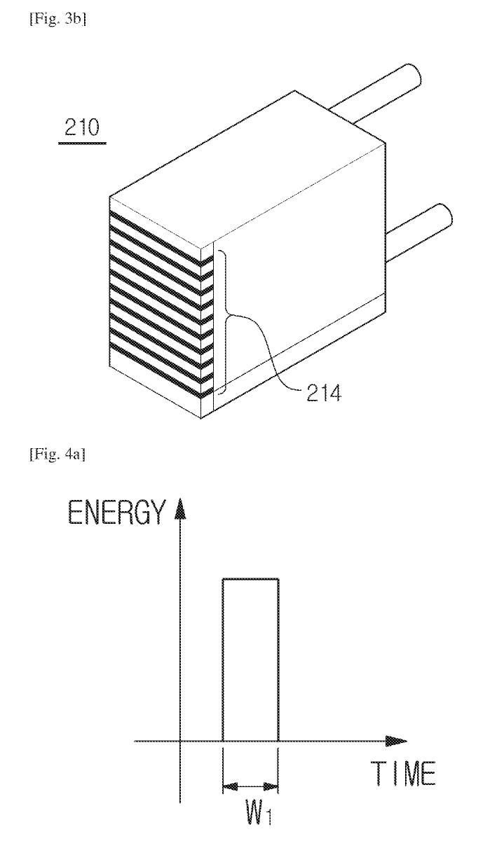

FIGS. 4A and 4B are graphs illustrating energy change of laser beams with respect to time.

FIG. 4A is a graph illustrating energy change of a pulsed laser beam with respect to time. A pulsed laser beam refers to a patterned laser beam with oscillation and stop periods. Temporal focusing properties of energy may considerably be improved using the pulsed laser beam. That is, as a pulse width that is a time period during which oscillation continues decreases, a pulse energy increases. In FIG. 4A, the pulse width is w1.

When pulsed laser beams are used to generate a photoacoustic image, accurate information regarding the object may be acquired based on high focusing properties of the pulsed laser beams. Furthermore, since the pulsed laser beam has repeated oscillation and stop periods with time, information regarding the object in the axial direction may be acquired using time delay of the acquired acoustic waves. In this regard, the axial direction refers to a proceeding direction of a laser beam into the object.

Thus, the photoacoustic imaging apparatus generally uses a pulsed laser beam having high energy focusing properties. The pulsed laser beam used herein may have a pulse width of 10 ns or less. However, since a pulsed laser beam generally has a pulse repetition rate of 20 Hz or less, it takes a long time to acquire information regarding the object to generate one photoacoustic image.

FIG. 4B is a graph illustrating energy change of a CW laser beam with respect to time. The CW laser beam is a continuously output laser beam without interruption regardless of time. Differently from the pulsed laser beam, the CW laser beam has the same output energy and does not have a pulse width or pulse repetition rate. Thus, even when the CW laser beam is applied to the photoacoustic imaging apparatus, problems caused by the pulsed laser beam are not caused.

However, it is difficult to directly apply the CW laser beam to the photoacoustic imaging apparatus. When the CW laser beam is projected onto the object, acoustic waves are continuously generated in response thereto. In this case, since it is difficult to identify origins of the acoustic waves generated in the object by the CW laser beam, an accurate photoacoustic image cannot be generated.

Thus, axial resolution with respect to the object needs to be improved by controlling the CW laser beam to have an energy waveform similar to that of the pulsed laser beam.

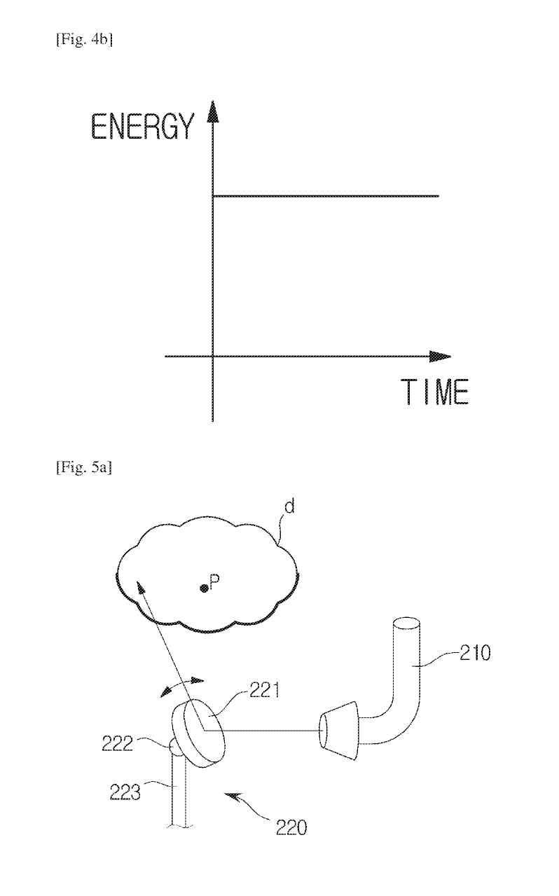

FIGS. 5A and 5B are diagrams for describing a method of controlling a CW laser beam to have an energy waveform similar to that of a pulsed laser beam using a deflection mirror.

Referring to FIG. 5A, the laser source 210 generates a CW laser beam. The generated CW laser beam is reflected by the reflective portion 221 of the deflection mirror 220 and proceeds in a direction marked as an arrow, thereby being projected onto the object d. Since the CW laser beam is projected onto a position unrelated to point P, an energy level of the CW laser beam applied to point P is 0 J.

Since the laser source 210 generates a CW laser beam, the CW laser beam reflected by the deflection mirror 220 may have the same energy level. The rotation portion 222 of the deflection mirror 220 may only rotate the reflective portion 221 by transmitting rotation force to the reflective portion 221. As a result, the rotating reflective portion 221 may reflect the CW laser beam in different directions as time passes.

FIG. 5B illustrates a case after a predetermined time period of t1 from FIG. 5A, and FIG. 5C illustrates a case after a predetermined time period of t2 from FIG. 5A. In FIGS. 5B and 5C, areas filled with slanted lines are areas of the object d onto which the CW laser beam is projected as time passes.

Referring to FIGS. 5A to 5C, while the deflection mirror 220 is rotated, the CW laser beam is projected onto point P for a predetermined time period. Generally, the CW laser beam having a uniform energy level is continuously projected onto the object d regardless of time. However, the CW laser beam may be projected onto point P only for a predetermined time period by using the deflection mirror 220.

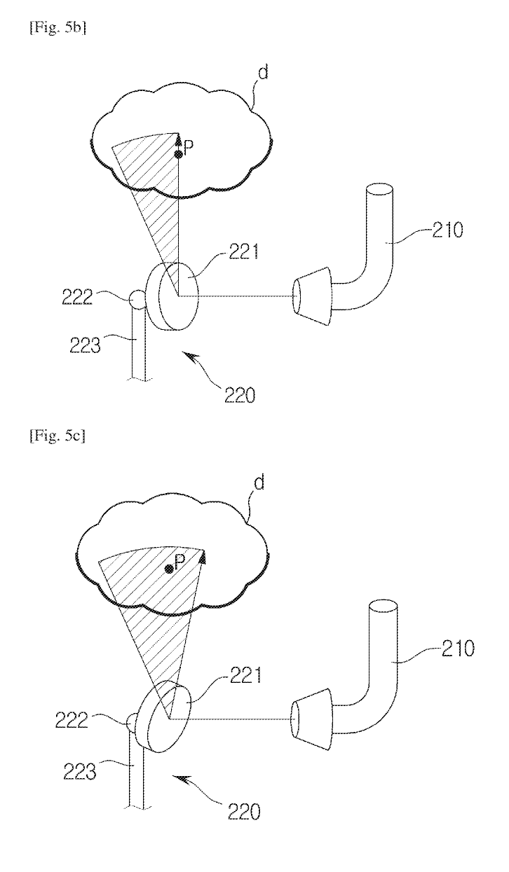

FIG. 6 is a graph illustrating energy change of a CW laser beam projected using a deflection mirror with respect to time. When the deflection mirror 220 is controlled as illustrated in FIGS. 5A to 5B, the CW laser beam is momentarily projected onto point P, and thus the energy level thereof has a positive real number. Since the CW laser beam is not projected to the other positions, the energy level of the CW laser beam applied to the other positions is 0 J.

As a result, the CW laser beam may have a discontinuous energy waveform as illustrated in FIG. 6. That is, even when the CW laser beam projected, the energy waveform may have a pulse width or a repetition rate.

When the CW laser beam is projected onto point P for a predetermined time period as illustrated in FIGS. 5A and 5B, an energy waveform having a pulse width of W2 as illustrated in FIG. 6 may be acquired. As described above with reference to FIG. 4A, a pulsed laser beam generally has a pulse width of 10 ns or less. As the pulse width decreases, energy focusing properties are improved, thereby generating a clearer photoacoustic image. Thus, the CW laser beam needs to be controlled such that the energy waveform of FIG. 6 has a pulse width within this range. Particularly, the pulse width is 10 ns or less.

The deflection mirror 220 may rotate the rotation portion 222 according to a predetermined rate of rotation. A time period w2 during which the CW laser beam is projected onto point P may be determined according to the rate of rotation of the rotation portion 222. Since the time period during which the CW laser beam is projected onto point P indicates the pulse width, the pulse width may be controlled to be 10 ns or less by setting the rate of rotation.

Particularly, when the rotation portion 222 transmits rotation force to the reflective portion 221 according to the predetermined rate of rotation, the reflective portion 221 may be rotated according to the predetermined rate of rotation. The rotating reflective portion 221 reflects the CW laser beam toward the object d, the time period during which the reflected laser beam is projected onto point P may be 10 ns or less.

The rate of rotation of the deflection mirror 220 may be set by a user via an input unit or may be set according to an internal calculation of the photoacoustic imaging apparatus so to control the pulse width as desired.

The deflection mirror 220 may set a rotation direction such that the reflected CW laser beam proceeds on the same plane. However, this is an exemplary embodiment of the present invention, and the rotation direction of the deflection mirror 220 is not limited thereto.

Referring back to FIG. 2, the transducer 230 may collect acoustic waves generated in the object onto which the CW laser beam is projected.

The transducer 230 may include piezoelectric ultrasonic transducers using piezoelectric effects of a piezoelectric material, magnetostrictive ultrasonic transducers using magnetostrictive effect of a magnetic element, or capacitive micromachined ultrasonic transducers (cMUTs) that receive acoustic waves using vibrations of hundreds and thousands micromachined thin films. Hereinafter, a description will be given of a piezoelectric transducer as the transducer.

A transducer 230 may include a piezoelectric layer to convert acoustic signals into electric signals, a matching layer disposed on the front surface of the piezoelectric layer, and a backing layer disposed on the back surface of the piezoelectric layer.

A phenomenon in which a voltage is generated when mechanical pressure is applied to a predetermined material, is referred to as a piezoelectric effect, and a material having such effect is referred to as a piezoelectric material. That is, the piezoelectric material is a material converting mechanical vibration energy into electric energy.

The piezoelectric layer is formed of a piezoelectric material and converts acoustic wave signals into electric signals.

The piezoelectric material constituting the piezoelectric layer may include a ceramic of lead zirconate titanate (PZT), a PZMT single crystal containing a solid solution of lead magnesium niobate and lead titanate, a PZNT single crystal containing a solid solution of lead zinc niobate and lead titanate, or the like.

The matching layer is disposed on the front surface of the piezoelectric layer to reduce difference in acoustic impedance between the piezoelectric layer and the object, thereby effectively transferring the acoustic waves generated in the piezoelectric layer to the object. The matching layer may include at least one layer and may be divided into a plurality of units with a predetermined width together with the piezoelectric layer by a dicing process.

The backing layer is disposed on the back surface of the piezoelectric layer, absorbs acoustic waves generated in the piezoelectric layer, blocks transmission of the acoustic waves toward the back surface of the piezoelectric layer, thereby preventing image distortion. The backing layer may include a plurality of layers in order to improve the attenuation or blocking effect of photoacoustic waves.

The transducer 230 may be arranged in a predetermined direction on one surface of the photoacoustic probe 200. The types of the photoacoustic probes 200 may be distinguished from each other based on arrangement.

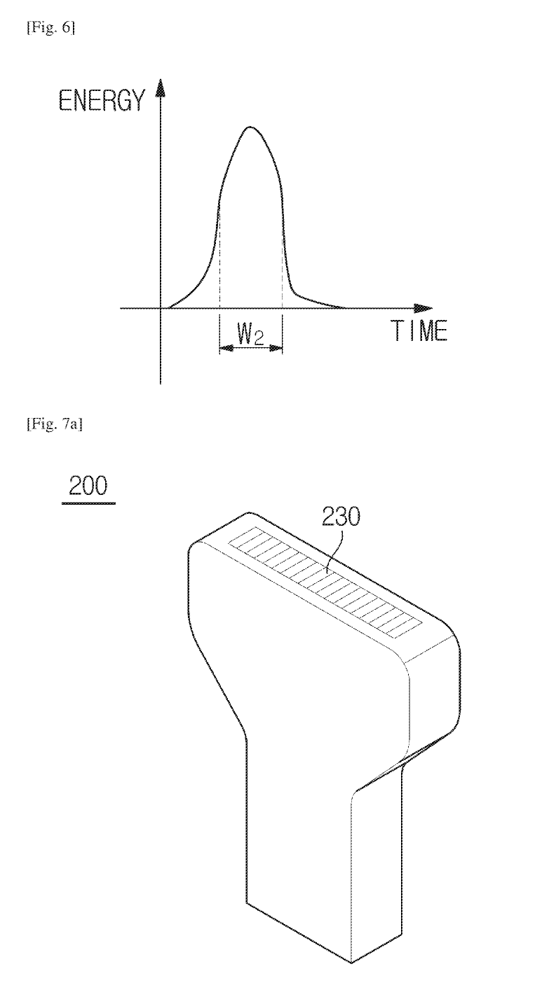

Referring to FIG. 7B, a convex array probe including the transducer 230 arranged on a curved surface may receive acoustic waves via the curved surface. Differently, a linear array probe of FIG. 7A including the transducer 230 arranged on a flat surface may receive acoustic waves via the flat surface.

However, the photoacoustic probe 200 is an exemplary embodiment of the present invention, and thus the photoacoustic imaging apparatus and the photoacoustic probe 200 used in a method of controlling the photoacoustic imaging apparatus according to embodiments of the present invention are not limited thereto. In addition, according to another embodiment of the photoacoustic imaging apparatus and the method of controlling the same, the photoacoustic probe 200 may be a two-dimensional (2D) array probe.

Although the photoacoustic probe including a piezoelectric ultrasonic transducer is described above, embodiments of the present invention are not limited thereto, and the transducer of the photoacoustic probe may vary so long as the transducer receives the acoustic waves.

The image processor 170 may generate a photoacoustic image based on the acoustic waves collected by the transducer 230. Techniques of generating a photoacoustic image based on acoustic waves are well known in the art, and thus detailed descriptions thereof will not be given herein.

The image processor 170 may be implemented as a hardware processor such as a central processing unit (CPU) or a graphics processing unit (GPU). However, image processing may also be implemented using hardware or software.

The display 160 may display the photoacoustic image generated by the image processor 170 on a screen. An examiner may perform a diagnosis on an internal region of the object d based on the photoacoustic image displayed on the display 160. Particularly, the examiner may check the health status of the object, e.g., a patient, or the existence of lesions from the photoacoustic image displayed on the screen of the display 160 and find a suitable treatment to improve the health status of the patient.

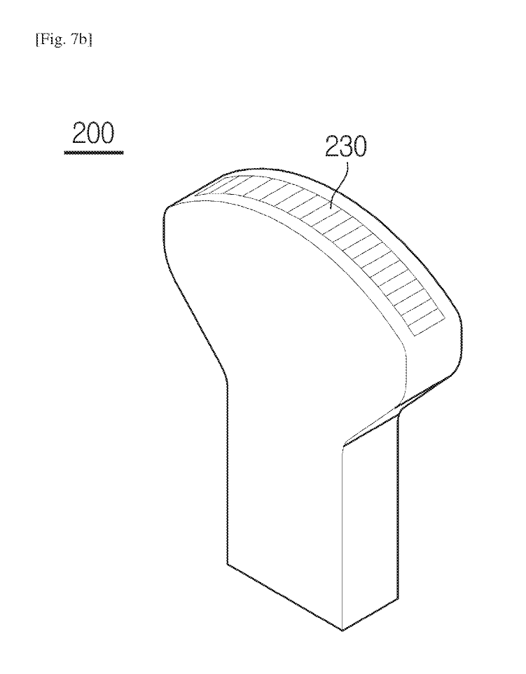

FIG. 8 is a control block diagram illustrating a photoacoustic imaging apparatus according to another embodiment of the present invention. The photoacoustic imaging apparatus may include a photoacoustic probe 200 to project a laser beam and receive acoustic waves, an image processor 170 to generate a photoacoustic image based on the acoustic waves received by the photoacoustic probe 200, and a display 160 to display the photoacoustic image generated by the image processor 170 on a screen.

In addition, the photoacoustic probe 200 may include a light delivery unit 240, which includes a laser source 210 to generate a CW laser beam and a deflection mirror 220 to reflect the CW laser beam toward an object while rotating, and a transducer 230 to collect acoustic waves generated in the object in response to the CW laser beam.

The photoacoustic imaging apparatus illustrated in FIG. 8 has the same configuration as that of the photoacoustic imaging apparatus illustrated in FIG. 2. However, in FIG. 8, the light delivery unit 240 includes the laser source 210 and the deflection mirror 220, and the photoacoustic probe 200 includes the light delivery unit 240 and the transducer 230.

Since the photoacoustic imaging apparatus may acquire acoustic waves by projecting the CW laser beam onto the object using a single element of the photoacoustic probe 200, the diagnosis process using the photoacoustic image may be simplified. Via simplification of the diagnosis process, uncertainty caused during generation of the photoacoustic image may be reduced, thereby creating an accurate photoacoustic image.

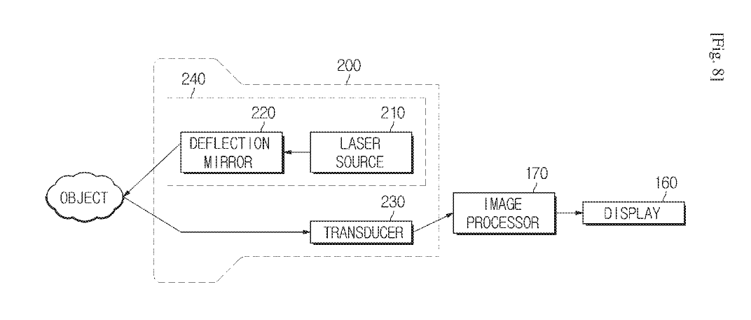

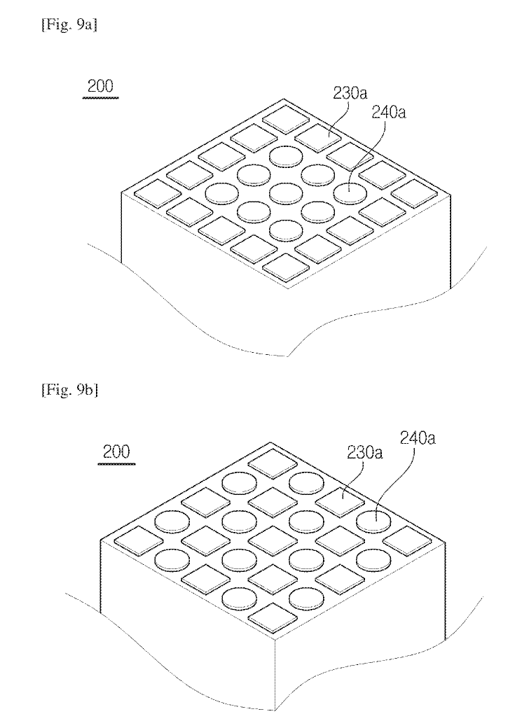

Since the photoacoustic imaging apparatuses of FIGS. 2 and 8 have the same configuration, descriptions of functions of each element will not be given herein. Hereinafter, the structure and operational principle of the photoacoustic probe 200 will be described in more detail with reference to FIGS. 9A to 9C and FIGS. 10A and 10B.

FIGS. 9A to 9C are diagrams illustrating various arrangements of a light delivery unit and a transducer on a photoacoustic probe.

The light delivery unit 240 and transducer 230 may be arranged on one surface of the photoacoustic probe 200. In the arrangement of the light delivery unit 240 and the transducer 230, each element thereof are respectively referred to as a light delivery element 240a and a transducer element 230a. In the photoacoustic imaging apparatus of FIG. 2, the laser source 210 projecting laser beams is separately formed from the transducer 230 receiving acoustic waves. However, the photoacoustic imaging apparatus of FIG. 8 projects laser beams and receives acoustic waves using the photoacoustic probe 200 as a single device.

FIG. 9A illustrates an example of a 2D array of the light delivery unit 240 and the transducer 230. In more detail, the transducer elements 230a receiving acoustic waves may be arranged at edges of the 2D array, and the light delivery elements 240a may be arranged at an inner portion of the transducer elements 230a.

FIG. 9B illustrates another example of the 2D array of the light delivery unit 240 and the transducer 230. In more detail, the transducer elements 230a are arranged to surround each of the light delivery elements 240a and the light delivery elements 240a are arranged to surround each of the transducer elements 230a. That is, the light delivery elements 240a and the transducer elements 230a may be arranged two-dimensionally and alternately.

FIG. 9C illustrates another example of the 2D array of the light delivery unit 240 and the transducer 230. Particularly, one-dimensionally arranged transducer elements 230a may constitute one column or row and one-dimensionally arranged light delivery elements 240a may constitute another column or row to be adjacent to the column or row of the transducer elements 230a. The 2D array of the transducer elements 230a and the light delivery elements 240a may be formed by alternately arranging columns or rows of the transducer elements 230a and the light delivery elements 240a which are one-dimensionally arranged as described above, respectively.

FIGS. 9A to 9C illustrate examples of the array of light delivery unit 240 and the transducer 230 on one surface of the photoacoustic probe 200, but arrangement method is not limited thereto. In addition, the light delivery unit 240 and the transducer 230 may be two-dimensionally arranged as illustrated in FIGS. 9A to 9C, but may also be one-dimensionally arranged differently therefrom.

FIG. 10A illustrates an external appearance of the light delivery element 240a. FIG. 10B illustrate an inner structure of the light delivery element 240a.

Referring to FIG. 10A, the light delivery element 240a may have a cylindrical shape. In addition, the light delivery element 240a may have an inner space in which the laser source 210 and the deflection mirror 220 are mounted. However, the cylindrical shape of the light delivery element 240a is an example, and the light delivery element 240a may have various shapes.

According to an embodiment, the light delivery element 240a may be formed of optical fiber in which cylindrical glass fiber disposed at the external surface.

The light delivery element 240a may project a laser beam generated therein outward through one surface. In FIG. 10A, arrows indicate proceeding directions of the projected laser beams. The laser beams generated therein may be projected in various directions.

Referring to FIG. 10B, the light delivery element 240a may include the laser source 210 generating the CW laser beam and disposed at one end thereof and the deflection mirror 220 reflecting the CW laser beam while rotating and disposed at the other end thereof.

The laser source 210 may be disposed at one end of the light delivery element 240a. The laser source 210 may generate the CW laser beam and project the generated CW laser beam. In this regard, since the projected laser beam may be split in various direction, a light emitting unit may include a lens 211 that focuses the laser beams in one direction.

The CW laser beam generated by the laser source 210 may proceed to the other end through the inner space of the light delivery element 240a. Here, the inside of the light delivery element 240a may have a structure suitable for reflection of the laser beam to facilitate the proceeding of the CW laser beam.

The deflection mirror 220 may be disposed at the other end of the light delivery element 240a. When the CW laser beam proceeds and reaches the deflection mirror 220, the deflection mirror 220 may reflect the CW laser beam while rotating. A pulse width of the CW laser beam may be determined according to the rate of rotation of the deflection mirror 220.

Meanwhile, the deflection mirror 220 disposed in the light delivery element 240a may be a micro mirror.

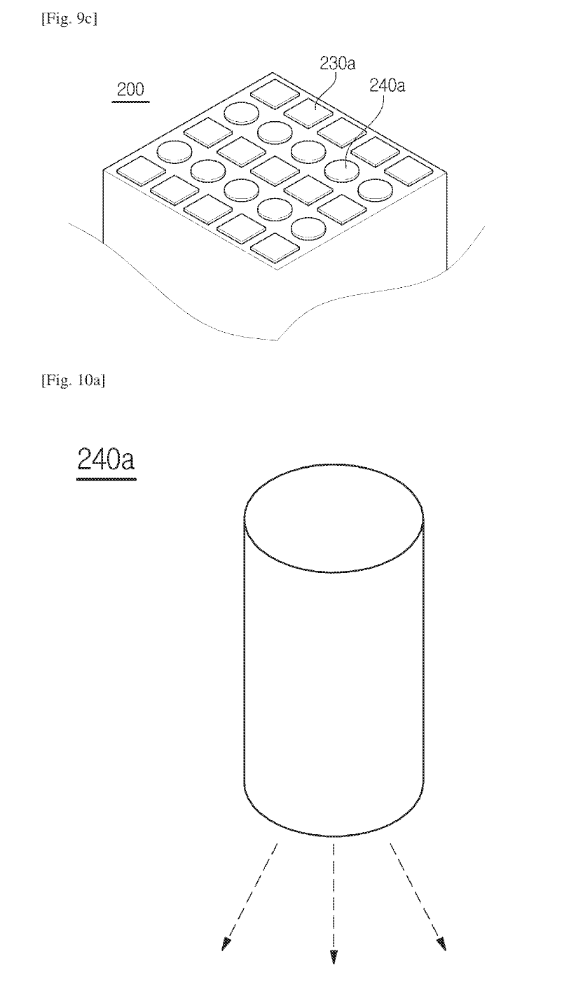

FIG. 11 is a control block diagram illustrating a photoacoustic imaging apparatus according to another embodiment of the present invention. The photoacoustic imaging apparatus may include a laser source 210 to generate a CW laser beam, a modulator 250, which modulates the CW laser beam generated by the laser source 210 to generate at least one frequency modulated continuous-wave (FMCW) laser beam and transmits the FMCW laser beam to a deflection mirror 220, the deflection mirror 220 to reflect the CW laser beam toward an object while rotating, a transducer 230 to collect acoustic waves generated in the object by the CW laser beam, and an image processor 170 to generate a photoacoustic image based on the collected acoustic waves.

FIG. 11 illustrates the same photoacoustic imaging apparatus as that of FIG. 2 further including the modulator 250. However, the photoacoustic imaging apparatus of FIG. 8 may further include a modulator 250. In this case, the modulator 250 may be provided inside or outside the photoacoustic probe 200.

The laser source 210 may generate the CW laser beam. Since the laser source 210 of FIG. 11 is the same as the laser sources 210 illustrated in FIGS. 2 and 8, and detailed descriptions thereof will not be given.

The modulator 250 may modulates frequency of the CW laser beam generated by the laser source 210. Frequency modulation refers to a method of modulating frequency of a carrier in accordance with amplitude of a signal wave.

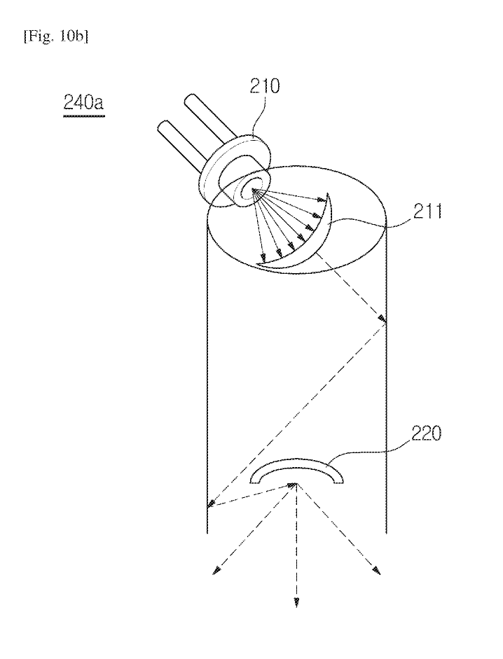

FIG. 12A illustrates a graph of energy change of a laser beam with respect to time on the left and a graph of energy change of the laser beam with respect to frequency on the right.

The CW laser beam may be controlled to have a waveform as illustrated in the right graph of FIG. 12a using the deflection mirror 220. That is, the CW laser beam may have an energy waveform similar to that of a pulsed laser beam when the rotating deflection mirror 220 reflects the CW laser beam while rotating.

The right graph of FIG. 12A may be obtained with respect to frequency from the left graph. Referring to the right graph, a center frequency of the energy waveform is fc. When the CW laser beam having the center frequency of fc is projected onto the object, an acoustic wave having the center frequency of fc may be generated in response thereto.

FIG. 12B illustrates a graph of energy change of a laser beam with respect to time on the left and a graph of energy change of frequency modulated laser beams therefrom with respect to frequency on the right.

Referring to the right graph of FIG. 12B, three energy waveforms having center frequencies of fc1, fc2, and fc3 are shown. Since a frequency of a carrier used in frequency modulation becomes a center frequency of a frequency-modulated waveform, referring to FIG. 12B, frequency modulations were performed three times.

As described above, the center frequency of the corresponding acoustic wave is determined according to the center frequency of the projected laser beam. Thus, when the FMCW laser beams as illustrated in the right graph of FIG. 12B are projected onto the object, the transducer 230 may receive acoustic waves having center frequencies of fc1, fc2, and fc3.

When the modulator 250 performs frequency modulation plural times by varying frequency, a plurality of FMCW laser beams may be generated. The modulator 250 may provide the plurality of FMCW laser beams generated as described above simultaneously or sequentially to the deflection mirror 220.

The deflection mirror 220 may reflect the FMCW laser beam to the object while rotating. Accordingly, the FMCW laser beam may have a waveform similar to that of a pulsed laser beam. The deflection mirror 220 is described above with reference to FIG. 2 or 8, and thus detailed descriptions thereof will not be given.

The transducer 230 may collect acoustic waves generated in the object by the FMCW laser beam. In this regard, the collected acoustic waves may have the same center frequencies as those of the CW laser beams.

The transducer 230 may include a plurality of different elements respectively collecting acoustic waves having different frequency bands or a plurality of the same elements each collecting all acoustic waves having different frequency bands.

Referring to FIG. 12B, the center frequencies of the projected laser beams are fc1, fc2, and fc3. Accordingly, the collected acoustic waves have the center frequencies of fc1, fc2, and fc3 as well. In this regard, some elements of the transducer 230 may have a frequency band suitable for collecting the acoustic waves having the center frequency of fc1. In addition, other elements of the transducer 230 may have a frequency band suitable for collecting the acoustic waves having the center frequency of fc2, and the other elements of the transducer 230 may have a frequency band suitable for collecting the acoustic waves having the center frequency of fc3.

Differently, the transducer 230 may include wideband elements capable of receiving acoustic waves in a wide frequency range. For example, referring to FIG. 12B, all elements of the transducer 230 may have a frequency band capable of receiving all photoacoustic waves respectively having the center frequencies of fc1, fc2, and fc3. In this regard, the wideband indicates that a frequency band of a receiving element is wider than a center frequency of an acoustic wave. In general, when a rate of the frequency band of the receiving element to the center frequency of the acoustic wave is 100% or greater, the frequency range of the receiving element may be regarded as a wideband element.

The image processor 170 classifies the collected acoustic waves according to at least one frequency band, generates at least one photoacoustic image based on the classified acoustic waves, synthesizes the generated at least one photoacoustic image to create one synthetic photoacoustic image.

When the elements of the transducer 230 have different frequency bands for receiving acoustic waves, the image processor 170 may classify the acoustic waves on the basis of the elements of the transducer 230. On the other hand, when the transducer 230 includes wideband elements, all of the collected acoustic waves may be classified on the basis of the frequency band.

Based on the classified acoustic waves, photoacoustic images may be generated. The number of photoacoustic images may be the same as the number of classified frequency band groups. Thus, at least one photoacoustic image may be generated.

When a plurality of photoacoustic images are generated, the photoacoustic images may be synthesized into one single synthetic photoacoustic image. Particularly, a synthetic photoacoustic image may be prepared by respectively applying weights to the photoacoustic images based on the frequency bands of the acoustic waves.

The acoustic waves have different attenuation rates according to frequency. Particularly, as frequency increases, an attenuation rate of an acoustic wave increases. Thus, although high-frequency acoustic waves may have relatively accurate information regarding the surface of the object, accuracy of acquired information regarding a deeper region of the object may decrease. On the contrary, low-frequency acoustic waves may have relatively accurate information regarding a deeper region of the object.

Based on such properties of the acoustic waves, in a photoacoustic image generated based on low-frequency acoustic waves, a weight may be applied to a portion corresponding to a deeper region of the object. Furthermore, in a photoacoustic image generated based on high-frequency acoustic waves, a weight may be applied to a portion corresponding to a region adjacent to the surface of the object. When a synthetic photoacoustic image is created by applying different weights to different portions according to the frequency bands of the acoustic waves, accuracy may be improved.

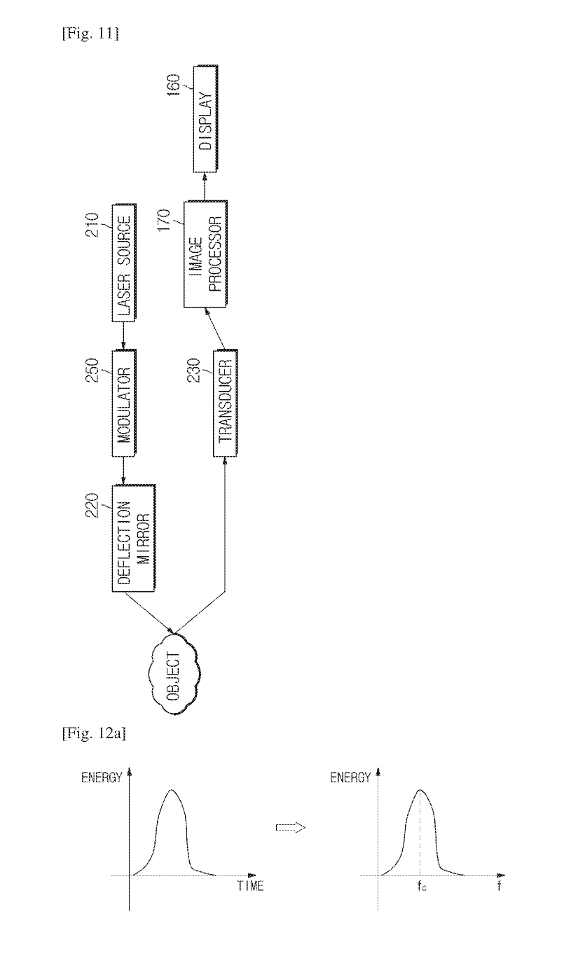

FIGS. 13A to 13C are diagrams for describing a method of applying weights to photoacoustic images according to an embodiment of the present invention.

A left graph of FIG. 13A illustrates an energy waveform of a CW laser beam, frequency of which is modulated using a carrier having a frequency of fc1. The FMCW laser beam generated by the modulator 250 has a center frequency of fc1.

An acoustic wave generated by the FMCW laser beam has the same center frequency of fc1. Among fc1, fc2, and fc3, fc1 is the lowest frequency, and thus a photoacoustic image generated based on the acoustic wave having the center frequency of fc1 may have accurate information regarding a deeper region of the object.

Thus, referring to a right diagram of FIG. 13A, a weight may be applied to a portion of the photoacoustic image corresponding to the deeper region of the object. FIG. 13B illustrates that a FMCW laser beam having the center frequency of fc2 is projected onto the object. Among fc1, fc2, and fc3, fc2 is the middle frequency, and thus a weight may be applied to a portion of the generated photoacoustic image corresponding to the central region of the object.

Similarly, referring to FIG. 13C, when a FMCW laser beam having the center frequency of fc3, which is the highest frequency, is projected onto the object, a weight may be applied to a portion of the generated photoacoustic image corresponding to a region adjacent to the surface of the object.

The image processor 170 may generate a synthetic photoacoustic image by synthesizing the photoacoustic images to which different weights are applied.

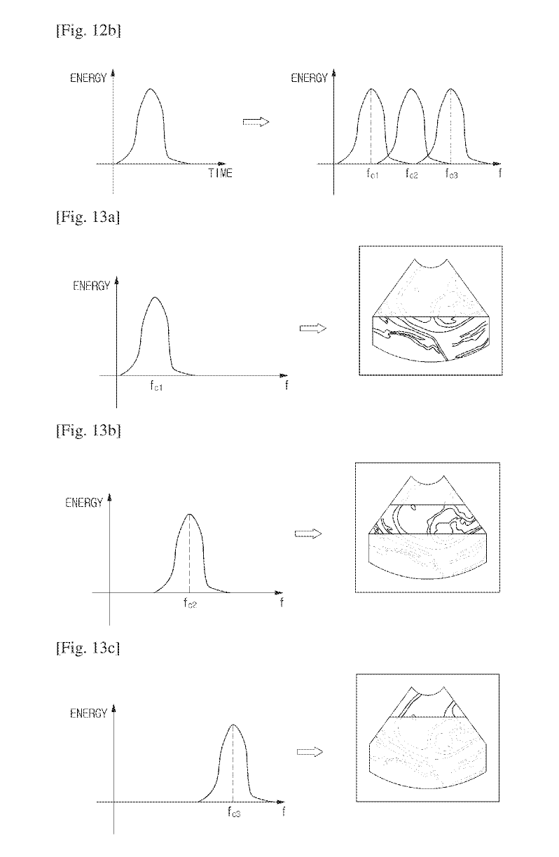

FIG. 14 is diagrams for describing a method of creating a synthetic photoacoustic image.

Referring to left diagrams of FIG. 14, different weights may respectively be applied to the photoacoustic images respectively generated on the basis of frequency band. A synthetic photoacoustic image may be generated using the photoacoustic images to which different weights are applied. A right diagram of FIG. 14 illustrates the synthetic photoacoustic image displayed on the display 160.

In the synthetic photoacoustic image, each of the portions of the photoacoustic images to which weights are applied may be emphasized. The synthetic photoacoustic image has excellent resolution in the depth direction, i.e., axial resolution, of the object since weights are applied to portions respectively having more accurate information regarding the object.

The generation of the synthetic photoacoustic image by the image processor 170 by applying weights to the photoacoustic images as described above with reference to FIGS. 13A to 13C and 14 is an exemplary embodiment to generate a synthetic photoacoustic image. Thus, the image processor 170 may apply weights to the photoacoustic images using various methods without being limited to the aforementioned embodiment and generate a synthetic photoacoustic image using the same.

The display 160 may display the synthetic photoacoustic image generated by the image processor 170 on the screen thereof. Alternatively, the non-synthesized photoacoustic images may respectively be displayed on the frequency basis or may be simultaneously displayed on a single screen.

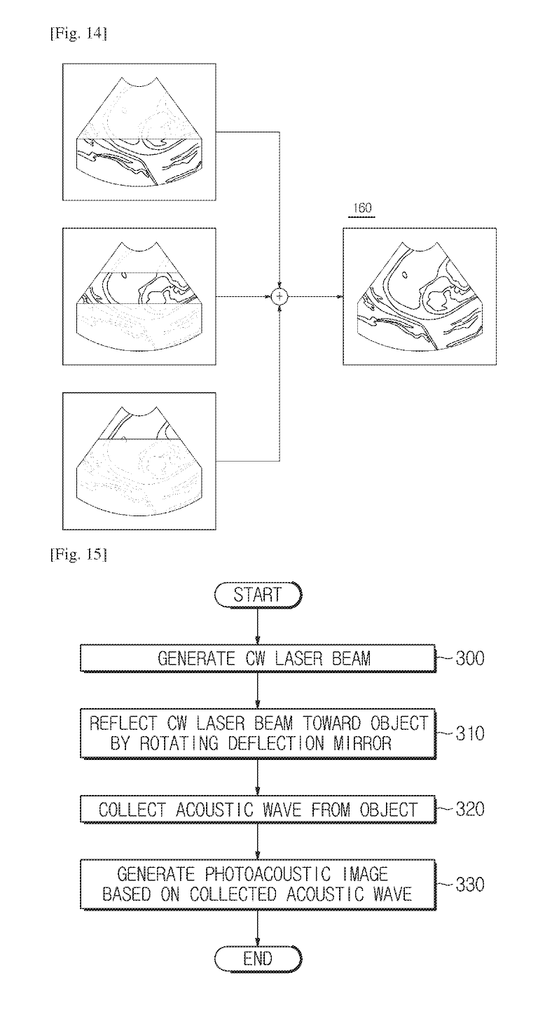

FIG. 15 is a flowchart illustrating a method of generating a photoacoustic image by projecting a CW laser beam according to an embodiment of the present invention.

First, a CW laser beam may be generated (300). Conventionally, a pulsed laser beam has been used to generate a photoacoustic image. In this case, however, a pulse repetition rate may decreases. Thus, a CW laser beam may be used to generate the photoacoustic image.

The generated CW laser beam may be reflected by the deflection mirror to proceed toward the object (310). In this case, the proceeding direction of the CW laser beam varies as the deflection mirror 220 rotates. The proceeding direction of the CW laser beam is changed using the deflection mirror 220 such that the CW laser beam is momentarily projected onto a predetermined region of the object. When a laser beam is momentarily projected, an energy waveform of the projected laser beam may be similar to that of a pulsed laser beam.

Here, the deflection mirror 220 may rotate according to a predetermined rate of rotation. Since a pulse width of the energy waveform of the laser beam projected to the predetermined region of the object is determined according to the rate of rotation of the deflection mirror 220, the rate of rotation may be determined by a user or an internal calculation of the photoacoustic apparatus.

Acoustic waves may be generated in the object by the CW laser beam projected onto the object. The generated acoustic waves may be collected by a transducer 230 (320). Based on the collected acoustic waves, a photoacoustic image may be generated (330).

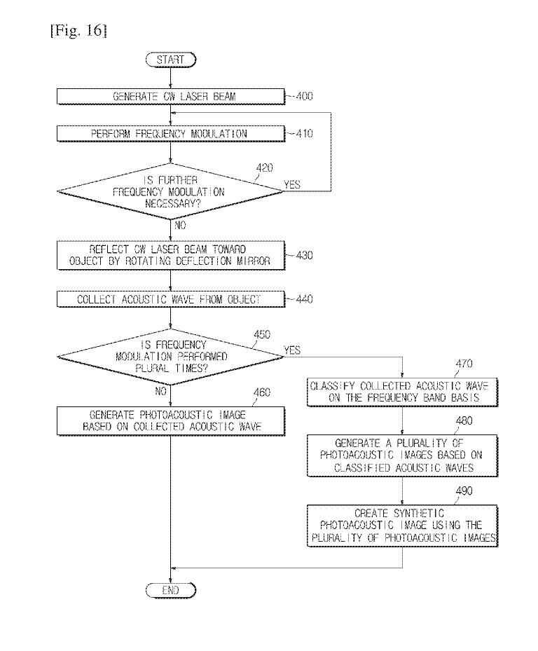

FIG. 16 is a flowchart illustrating a method of generating a photoacoustic image by projecting a CW laser beam according to another embodiment of the present invention.

First, a laser source generates a CW laser beam (400). The CW laser beam is generated for the reasons described above with reference to FIG. 15.

The generated CW laser beam is subjected to frequency modulation to generate a frequency modulated continuous-wave (FMCW) laser beam (410). A frequency of a carrier used in the frequency modulation becomes a center frequency of the FMCW laser beam.

Necessity to further perform frequency modulation is checked (420). If required, frequency modulation may further be performed using another carrier. In this case, a plurality of FMCW laser beams may be generated.

When frequency modulation is sufficiently performed, the FMCW laser beams are reflected by the deflection mirror toward the object (430). The deflection mirror 220 may be used to project the laser beam to the object for the reasons described above with reference to FIG. 15.

In this regard, when frequency modulation is performed plural times, a plurality of generated FMCW laser beams may simultaneously be reflected by the deflection mirror 220. Alternatively, the plurality of generated FMCW laser beams may sequentially be reflected by the deflection mirror 220.

When the FMCW laser beams are projected onto the object, the object may thermo-elastically expand, thereby generating acoustic waves. The transducer 230 may collect the generated acoustic waves (440). In this case, the transducer 230 may include elements respectively receiving acoustic waves having different frequency bands. Alternatively, the transducer 230 may include wideband elements receiving all acoustic waves having different frequency bands.

When the acoustic waves are collected, it is identified whether frequency modulation is performed plural times in order to generate a photoacoustic image (450). When the frequency modulation is performed once, a photoacoustic image is generated based on the collected acoustic waves (460). However, when the frequency modulation is performed plural times, a process of creating a synthetic photoacoustic image is performed.

Particularly, the collected acoustic waves may be classified on the frequency band basis (470). Then, a plurality of photoacoustic images may be generated based on the classified acoustic waves (480). Here, the number of generated photoacoustic images may be identical to the number of frequency modulation of the CW laser beam.

A synthetic photoacoustic image may be created using the plurality of photoacoustic images (490). To this end, different weights may respectively be applied to the photoacoustic images. Particularly, when the weights are applied in consideration of attenuation properties of the acoustic waves according to frequency, axial resolution with regard to the object may be improved. The plurality of photoacoustic images to which different weights are applied may be used to create one synthetic photoacoustic image, and the synthetic image may be displayed on the display 160 to allow an examiner to diagnose the inside of the object.

As is apparent from the above description, according to the photoacoustic imaging apparatus and the method of controlling the same according to an embodiment of the present invention, a photoacoustic image may have increased frame rate by projecting a CW laser beam onto an object.

Although a few embodiments of the present invention have been shown and described, it would be appreciated by those skilled in the art that changes may be made in these embodiments without departing from the principles and spirit of the invention, the scope of which is defined in the claims and their equivalents.

1. A photoacoustic imaging apparatus comprising:
a laser source to generate a continuous-wave laser beam;
a deflection mirror to reflect the continuous-wave laser beam toward an object while rotating;
a transducer to collect acoustic waves generated in the object by the continuous-wave laser beam; and
an image processor to generate a photoacoustic image based on the collected acoustic waves.

2. The photoacoustic imaging apparatus according to claim 1, further comprising a modulator to modulate the continuous-wave laser beam generated by the laser source into at least one frequency modulated continuous-wave laser beam and provide the frequency modulated continuous-wave laser beam to the deflection mirror.

3. The photoacoustic imaging apparatus according to claim 2, wherein when a plurality of frequency modulated continuous-wave laser beams are generated, the modulator simultaneously or sequentially provides the plurality of frequency modulated continuous-wave laser beams to the deflection mirror.

4. The photoacoustic imaging apparatus according to claim 2, wherein when a plurality of frequency modulated continuous-wave laser beams are generated, the transducer comprises a plurality of different elements respectively collecting acoustic waves having different frequency bands or a plurality of the same elements collecting all acoustic waves having different frequency bands.

5. The photoacoustic imaging apparatus according to claim 2, wherein when a plurality of frequency modulated continuous-wave laser beams are generated, the image processor creates a synthetic photoacoustic image by classifying the collected acoustic waves on the basis of a plurality of frequency bands, generating a plurality of photoacoustic images based on the classified acoustic waves, and synthesizing the plurality of generated photoacoustic images.

6. The photoacoustic imaging apparatus according to claim 5, wherein the image processor creates the synthetic photoacoustic image by applying different weights respectively to the plurality of generated photoacoustic images.

7. The photoacoustic imaging apparatus according to claim 1, wherein the deflection mirror rotates according to a predetermined rate of rotation.

8. The photoacoustic imaging apparatus according to claim 1, wherein the laser source comprises a single laser diode or a plurality of laser diodes arranged in a predetermined direction.

9. A method of controlling a photoacoustic imaging apparatus, the method comprising:
generating a continuous-wave laser beam;
reflecting the continuous-wave laser beam toward the object by projecting the continuous-wave laser beam onto a rotating deflection mirror;
collecting acoustic waves generated in the object by the continuous-wave laser beam; and
generating a photoacoustic image based on the collected acoustic waves.

10. The method according to claim 9, wherein the generating of the continuous-wave laser beam comprises modulating the generated continuous-wave laser beam into at least one frequency modulated continuous-wave laser beam.

11. The method according to claim 10, wherein when a plurality of frequency modulated continuous-wave laser beams are generated, the projecting of the continuous-wave laser beam onto the rotating deflection mirror comprises simultaneously or sequentially projecting the plurality of frequency modulated continuous-wave laser beams onto the deflection mirror.

12. The method according to claim 10, wherein when a plurality of frequency modulated continuous-wave laser beams are geneated, the collecting of the acoustic waves comprises respectively collecting acoustic waves having different frequency bands using a plurality of different transducer elements, or collecting all acoustic waves having different frequency bands using a plurality of the same transducer elements.

13. The method according to claim 10, wherein when a plurality of frequency modulated continuous-wave laser beams are geneated, the generating of the photoacoustic image comprises:
classifying the collected acoustic waves on a basis of a plurality of frequency bands;
generating a plurality of photoacoustic images based on the classified acoustic waves, and
creating a synthetic photoacoustic image by synthesizing the plurality of generated photoacoustic images.

14. The method according to claim 13, wherein the creating of the synthetic photoacoustic image is performed by creating a synthetic photoacoustic image by respectively applying different weights to the plurality of generated photoacoustic images.

15. The method according to claim 9, wherein the deflection mirror rotates according to a predetermined rate of rotation.

16. The method according to claim 9, wherein the creating of the continuous-wave laser beam is performed using a laser source comprising a single laser diode or a plurality of laser diodes arranged in a predetermined direction.
